(19) United States
(12) Patent Application Publication
(10) Pub. No.: US 2009/0263465 A1
(43) Pub. Date: Oct. 22, 2009

(54) Biopolymer-Bioengineered Cell Sheet Construct

(75) Inventors: Ging-Ho HSIUE, Hsinchu (TW); Jui-Yang Lai, Hsinchu (TW)

Correspondence Address: BACON & THOMAS, PLLC, 625 SLATERS LANE, FOURTH FLOOR, ALEXANDRIA, VA, 22314-1176, US

(73) Assignee: National Tsing Hua University, Hsinchu (TW)

(21) Appl. No.: 12/494,465

(22) Filed: Jun. 30, 2009

Related U.S. Application Data
(62) Division of application No. 11/508,241, filed on Aug. 23, 2006.

(51) Int. Cl. A61F 2/00; A61K 35/12

(52) U.S. Cl. 424/426; 424/93.7

(57) ABSTRACT

A biopolymer-bioengineered human corneal endothelial cell (HCEC) sheet construct for reconstructing corneal endothelium in a patient is recited. The construct includes a biopolymer carrier which is bioresorable and deformable; and a bioengineered cell sheet containing a monolayer of interconnected HCECs with substantially uniform orientation. The bioengineered cell sheet is attached to a surface of the biopolymer carrier with apical surfaces of the HCECs facing said carrier.

### FIELD OF THE INVENTION

The present invention is related to a biopolymer-bioengineered cell sheet construct, and in particular to a biopolymer-bioengineered human corneal endothelial cell sheet construct for reconstructing corneal endothelium in a patient.

### BACKGROUND OF THE INVENTION

Human corneal endothelial cells (HCECs) maintain corneal clarity by a barrier function and pump-leak mechanism. Regarded as nonproliferative in vivo, HCECs decrease with aging and other factors such as inflammation, contact lens wearing, and trauma. Full-thickness corneal transplantation (penetrating keratoplasty, PK) is currently the common way to treat corneas that are opacified due to endothelial dysfunction. In these cases, considering insufficient supplies of donor corneas and complications of PK, there would be a substantial advantage in being able to replace the endothelium alone by delivering cultured HCECs to the recipient.

Corneal endothelial cell transplantation has been attempted to repopulate rabbit cornea with unhealthy endothelium by directly injecting a cell suspension into the anterior chamber. However, this trial has been limited because of only scattered clumps of endothelial cells randomly attach to the targeted cornea, and other normal ocular tissues such as iris and lens. In recent years, numerous investigators have reported a method to transplant corneal endothelial cells by seeding and cultivating them on different carriers made of either natural tissue materials [Lange T M, Wood T O, McLaughlin B J. Corneal endothelial cell transplantation using Descemet's membrane as a carrier. J Cataract Refract Surg. 1993; 19:232-235; Ishino Y, Sano Y, Nakamura T, et al. Amniotic membrane as a carrier for cultivated human corneal endothelial cell transplantation. Invest Opthalmol Vis Sci. 2004; 45:800-806] or artificial polymeric materials [Jumblatt M M, Maurice D M, Schwartz B D. A gelatin membrane substrate for the transplantation of tissue cultured cells. Transplantation. 1980; 29:498-499; Mohay J, Lange T M, Soltau J B, Wood T O, McLaughlin B J. Transplantation of corneal endothelial cells using a cell carrier device. Cornea. 1994; 13:173-182; Mimura T, Yamagami S, Yokoo S, et al. Cultured human corneal endothelial cell transplantation with a collagen sheet in a rabbit model. Invest Opthalmol Vis Sci. 2004; 45:2992-2997]. Although a monolayered architecture of cultured cells was maintained, the intraocular grafting of these engineered tissue replacements may possibly cause problems such as unstable attachment of cell carrier membrane to host corneal stroma, and fibroblastic overgrowth between the membrane and stroma (McCulley J P. Maurice D M, Schwartz B D. Corneal endothelial transplantation. Opthalmology. 1980; 87:194-201]. The principal problems with a method using cell carrier membranes are due to the permanent residence of these foreign materials in the host.

Cultivation of adult HCECs from older donors has been proven to be difficult [Senoo T, Joyce N C. Cell cycle kinetics in corneal endothelium from old and young donors. Invest Opthalmol Vis Sci 2000; 41: 660]. Chen at al. have developed a growth factors-enriched medium to succeed in mass culturing untransformed adult HCECs, and they have also shown that the cultivated confluent HCECs could grow with a cell polarity, the tight junction and microvilli on the apical surface by transmission electron microscopy [Chen K H, Azar D, Joyce N C. Transplantation of adult human corneal endothelium ex vivo: a morphologic study. Cornea 2001; 20: 731.

To obtain the transplantable HCEC sheets with intact cellular arrangement and organization, Yamada et al. have established a strategy based on the techniques of cell sheet engineering, which is used for harvesting in vitro cultivated cell sheets through external temperature modulation of thermo-responsive culture substrates [Yamada N, Okano T, Sakai H, Karikusa F, Sawasaki Y, Sakurai Y. Thermo-responsive polymeric surfaces; control of attachment and detachment of cultured cells. Macromol Rapid Commun 1990; 11: 571]. Yamada et al. have also reported that the cultivated cells could adhere and proliferate on the hydrophobic poly(N-isopropylacrylamide) (PNIPAAm)-grafted surfaces at 37° C., and detached from the hydrophilic surfaces due to abrupt hydrated transition of polymer chains when the culture temperature was lowered to a level below the lower critical solution temperature of PNIPAAm. Recently, this novel technology has been proven to be effective for cardiac tissue repair [Shimizu T, Yamato M, Isoi Y, et al. Fabrication of pulsatile cardiac tissue grafts using a novel 3-dimensional cell sheet manipulation technique and temperature-responsive cell culture surfaces. Circ Res 2002; 90: e40] and corneal epithelial reconstruction [Nishida K, Yamato M, Hayashida Y, et al. Functional bioengineered corneal epithelial sheet grafts from corneal stem cells expanded ex vivo on a temperature-responsive cell culture surface. Transplantation 2004; 77: 379; Nishida K, Yanato M, Hayashida Y, et al. Corneal reconstruction with tissue-engineered cell sheets composed of autologous oral mucosal epithelium. N Engl J Med 2004; 351: 1187; and Hayashida Y, Nishida K, Yamato M, et al. Ocular surface reconstruction using autologous rabbit oral mucosal epithelial sheets fabricated ex vivo on a temperature-responsive culture surface. Invest Opthalmol Vis Sci 2005; 46: 1632].

### SUMMARY OF THE INVENTION

A primary objective of the present invention is to provide a biopolymer-bioengineered cell sheet construct, which comprises a biopolymer carrier which is bioresorable and deformable; and a bioengineered cell sheet comprising a monolayer or multilayer of interconnected cells with substantially uniform orientation, wherein said bioengineered cell sheet is attached to a surface of said carrier with apical surfaces of the cells facing said carrier.

Preferably, said bioengineered cell sheet further comprises an extracellular matrix (hereinafter abbreviated as ECM) distributed at basal surfaces of said cells.

Preferably, said cells are human corneal endothelial cells.

Alternatively, said cells are human corneal epithelial cells.

Preferably, said biopolymer carrier is made of poly(amino acids), gelatin, collagen, polysaccharide, hyaluronan, chitosan, alginate, agarose, poly(α-hydroxy acid), or a mixture thereof, and gelatin is more preferable. Preferably said gelatin has a weight-average molecular weight of 10,000 to 200,000 Dalton, more preferably 50,000 to 100,000 Dalton, and has an isoelectric point of 1-10, and more preferably 5-9.

In one of the preferred embodiments of the present invention, the gelatin used has a weight-average molecular weight of 100,000 Dalton, and an isoelectric point of 5. Preferably, said gelatin is negatively charged.

Preferably, said biopolymer carrier has a thickness of 0.5-1.0 mm and a diameter of 5-10 mm, and has a water content of 10-90%, based on the dry weight of the biopolymer carrier. More preferably, said biopolymer carrier has a water content of less than 40%, based on the dry weight of the biopolymer carrier, when said bioengineered cell sheet is attached to the surface of said carrier, and said carrier becomes swollen and the water content thereof becomes at least 1.5-fold when the carrier is surround by an aqueous solution for a period of 5 minutes or more.

Another objective of the present invention is to provide a method for reconstructing corneal endothelium in a patient, which comprises implanting a biopolymer-bioengineered cell sheet construct into an anterior chamber of a cornea of the patient, wherein the construct comprises a biopolymer carrier which is bioresorable and deformable; and a bioengineered cell sheet comprising a monolayer or multilayer of interconnected endothelial cells with substantially uniform orientation, wherein said bioengineered cell sheet is attached to a surface of said carrier with apical surfaces of the endothelial cells facing said carrier, wherein the biopolymer-bioengineered cell sheet construct is implanted into the anterior chamber with basal surfaces of said endothelial cells of said bioengineered cell sheet contacting a posterior surface of the cornea.

Preferably, the method of the present invention further comprises removing unhealthy endothelium from the posterior surface of the cornea of the patient before said implanting.

Preferably, said implanting comprises forming an incision at a limbus of the cornea; inserting the biopolymer-bioengineered cell sheet construct through the incision into the anterior chamber; and closing the incision by suturing, so that the biopolymer-bioengineered cell sheet construct is enclosed in the anterior chamber, wherein the carrier will become swollen by aqueous humor in the anterior chamber, creating a pressure pressing the bioengineered cell sheet against the posterior surface of the cornea, and the carrier is eventually biodegraded in situ while an endothelial sheet is regenerated on the denuded posterior surface of the cornea. More preferably, the method of the present invention further comprises removing unhealthy endothelium from the posterior surface of the cornea before inserting the biopolymer-bioengineered cell sheet construct into the anterior chamber.

The present invention presents a novel technique to transplant cultivated HCECs as a cell sheet directly onto corneas without permanent residence of cell carriers in the host. Additionally, the transplanted HCEC sheet was demonstrated, along with a normal morphology and the function maintaining the corneal deturgescence.

### BRIEF DESCRIPTION OF THE DRAWINGS

FIGS. 1A to 1E are schematic views showing a novel strategy for corneal endothelial reconstruction with bioengineered cell sheets of the present invention, wherein FIGS. 1A to 1C show the cultured HCEC sheet 20 is harvested via temperature modulation of the thermo-responsive surface of the substrate 10, FIGS. 1D to 1E shows the delivering to corneal posterior surfaces without endothelium using a biodegradable biopolymer carrier 30, adhesive gelatin hydrogel discs, FIG. 1F shows the swelling of the carrier 30, and FIG. 1G shows the biodegradation of the carrier 30 and the transplanted HCEC sheet 20 with uniformly proper polarity being attached and integrated onto the denuded cornea 40 to allow regeneration of the endothelial sheet.

FIGS. 2A to 2F are photographs showing assessments of in vitro characteristics of the harvested HCEC sheet. FIGS. 2A and 2B are phase-contrast micrographs showing after 1 week of cultivation on the PNIPAAm-grafted surface at 37° C., confluent HCEC cultures were polygonal. By a further incubation for 2 weeks, the detachment of monolayered HCECs exhibited a sheet-like movement. Scale bars, 100 μm. FIG. 2C shows that the cultivated HCEC sheet was detached as a cell sheet with a size of around 0.75 cm² after 45 min of incubation at 20° C. Scale bar, 5 mm. FIG. 2D shows that most of the monolayered cells were viable (green fluorescence). Fewer dead cells (red fluorescence) were identified by Live/Dead staining. Scale bar, 50 μm. FIG. 2E is a SEM photograph showing multiple cellular interconnections (fine arrow) within the HCEC sheets. A layer of ECM (large arrow) was distributed at the basal cell surface. Scale bar, 50 μm. FIG. 2F is a SEM photograph showing a typical discontinuous tight junction was detected by immunostaining for ZO-1 protein (arrow), which indicated barrier formation. Scale bar, 10 μm.

FIG. 3 shows the time course of dissolution degree of various gelatin hydrogel discs after incubation in BSS at 34° C., wherein an asterisk indicates statistically significant differences (*p<0.05; n=3) for the mean value of dissolution degree compared to value at previous time point, and a gelatin sample with an IEP of “x” and a weight-average MW of “y” kDa is designated as G-x-y.

### DETAILED DESCRIPTION OF THE INVENTION

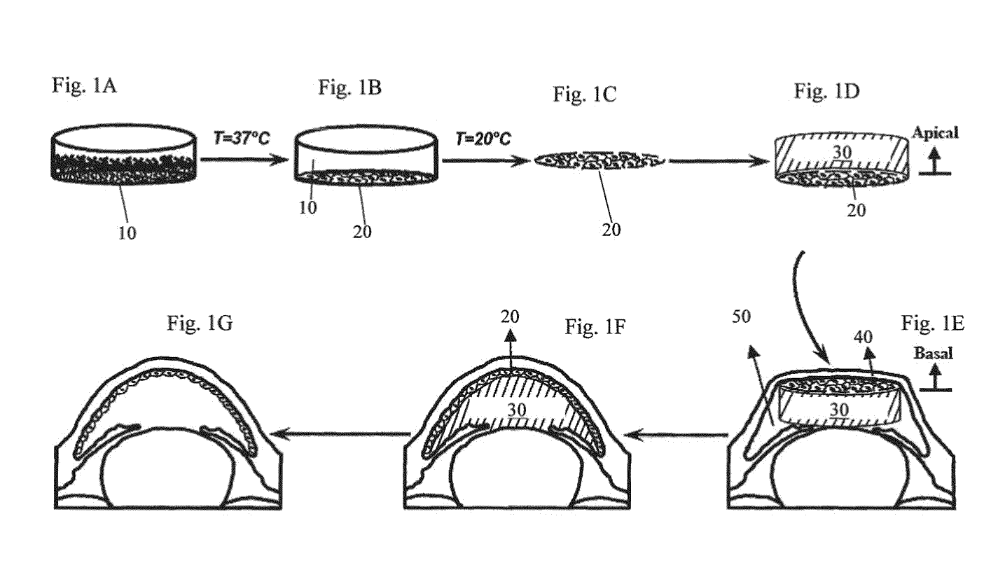

In the present invention, we present a novel therapy technique to transplant cultured HCECs as a cell sheet for reconstituting a corneal endothelial sheet in vivo. As shown in FIGS. 1A to 1E, an intelligent cell culture substrate 10 is prepared by surface modification with a thermo-responsive polymer such as poly(N-isopropylacrylamide) (abbreviated as PNIPAAm hereinafter). Untransformed HCECs derived from older individuals are further cultivated on the thermo-responsive surface of the substrate. Upon confluence, the tissue-engineered HCEC sheet 20 is harvested via thermal stimulus. In addition, a biopolymer carrier 30 preferably with multiple properties such as transparent, cell-adhesive, deformable, biodegradable, bioabsorbable, and biocompatible is exerted to provide a temporary support construct during and after in vivo delivery of the HCEC sheet 20 to recipient cornea 40 denuded of endothelium. The tissue-engineered HCEC sheet 20 is attached to a surface of said carrier 30 with apical surfaces of the endothelial cells facing said carrier 30. The construct is implanted into the anterior chamber 50 with basal surfaces of said endothelial cells of said HCEC sheet 20 contacting a posterior surface of the cornea 40. Without permanent residence of the carriers 30 in the host, the transplanted HCEC sheets 20 were demonstrated in the following experiments, along with the normal morphology and function maintaining the corneal deturgescence.

### Experiments:

### HCEC Cultivation

The following materials were purchased commercially for use in the cell cultivation. Human eye bank corneas were from National Disease Research Interchange (Philadelphia, Pa., USA). Optisol-GS was from Bausch & Lomb (Rochester, N.Y., USA). OPTI-modified Eagle's medium (OPTI-MEM), Medium 199 (M199), trypsin/ethylenediaminetetraacetic acid (0.05% trypsin/0.53 mM EDTA), gentamicin, and Hanks' balanced salt solution (HBSS; pH 7.4) were from GIBCO-BRL Life Technologies (Grand Island, N.Y., USA). Antibiotic/antimycotic solution (10000 U/mL of penicillin, 10 mg/mL of streptomycin and 25 μg/mL of amphotericin B) and fetal bovine serum (FBS) were from Biological Industries (Kibbutz Beit Haemek, Israel). Dispase II (2.4 U/mL) was from Roche Diagnostics (Indianapolis, Ind., USA). Dulbecco's phosphate-buffered saline (DPBS; pH 7.4) was from Biochrom AG (Berlin, Germany). Bovine pituitary fibroblast growth factor (FGF), ascorbic acid, human lipids, calcium chloride, chondroitin sulfate and RPMI-1640 vitamins solution were from Sigma-Aldrich (St. Louis, Mo., USA). Human recombinant epidermal growth factor (EGF) was from Upstate Biotechnology (Lake Placid, N.Y., USA). Nerve growth factor (NGF) was from Biomedical Technologies (Stoughton, Mass., USA). Sodium hyaluronate was from Lifecore Biomedical (Chaska, Minn., USA).

Twenty-five corneas from human donors (age, 55-80 years) stored in Optisol-GS at 4° C. were used. Endothelial cell counts were more than 2000 cells/mm². Criteria for exclusion of corneas from these studies included low endothelial cell density, history of endothelial dystrophy, and ocular inflammation or disease.

For the harvest of endothelial cells, each cornea tissue was placed in a Petri dish containing M199 and 50 μg/mL of gentamicin. Under a dissecting stereomicroscope (MZ75; Leica Microsystems, Wetzlar, Germany), Descemet's membrane with the attached endothelium was aseptically stripped from the stroma and washed three times with DPBS. The Descemet's membrane-corneal endothelium complex was then digested using a 1.2 U/mL of dispase II in HBSS for 1 hour at 37° C. The endothelial cells were further dislodged from Descemet's membrane by vigorous disruption with a flame-polished pipette, and a cell pellet was collected via centrifugation (1000 rpm, 4° C., 5 min). Thereafter, HCECs were resuspended and cultured in regular growth medium that consists of OPTI-MEM supplemented with 15% FBS, 40 ng/mL of FGF, 5 ng/mL of EGF, 20 ng/mL of NGF, 20 μg/mL of ascorbic acid, 0.005% human lipids, 0.2 mg/mL of calcium chloride, 0.08% chondroitin sulfate, 100 μg/mL of hyaluronan, 1% RPMI-1640 vitamins solution, 50 μg/mL of gentamicin, and 1% antibiotic/antimycotic solution.

Cell cultures were incubated in a humidified atmosphere of 5% CO₂ at 37° C. Medium was changed every other day. Confluence was reached after 1 week in culture. Cells were then subcultured by treating with trypin/EDTA for 2 min and seeded at a 1:2-1:4 split ratio. Only second-passage HCECs were used during all experiments.

### Preparation of Thermo-Responsive Culture Substrates

A two-step method, based on plasma-induced graft polymerization, was proposed to develop thermo-responsive polymeric surfaces for temperature-controlled cell cultivation and separation. At the first stage of this method, PAAc was introduced onto peroxidized polyethylene (PE) substrates by plasma activation and thermal graft polymerization. At the second stage, carboxyl groups on the AAc-grafted chains act as reaction sites for photografting polymerization of NIPAAm. Low-density PE dishes (35 mm in diameter) from USI Far East (Taipei, Taiwan, ROC) were ultrasonically cleaned in ethanol for 1 hour and then dried at room temperature before usage. Acrylic acid (AAc) (Merck, Whitehouse Station, N.J., USA) was purified by distillation under vacuum. NIPAAm (Acros Organics, Fairlawn, N.J., USA) was purified by recrystallization from n-hexane and dried at room temperature in vacuum.

A glow discharge reactor (Model PD-2 plasma deposition system) with a bell jar type reactor cell manufactured by Samco (Kyoto, Japan) was used. Plasma treatment of the PE substrates was carried out as follows. PE substrates were placed over the electrode. The pressure in the bell jar was reduced to 50 mtorr, which was followed by introduction of Ar gas into the bell jar and evacuation to 50 mtorr. This process was repeated three times. Plasma was next generated at 120 W, and the substrates were exposed to plasma for 90 seconds. After the plasma treatment, oxygen gas was introduced into the bell jar reactor at a flow rate of 200 mL/min for 20 min. The treated samples were kept under 1 atm of oxygen. After the exposure to oxygen gas, the plasma-treated PE substrates were placed in glass chambers containing a monomer solution which was prepared at a 12.5% of AAc and Mohr's salt (Ammonium-Fe(II)-sulfate purchased from Aldrich Chemical (Milwaukee, Wis., USA)). For thermal graft polymerization, the chambers were sealed after being degassed three times using nitrogen gas, and the reaction was performed at 70° C. with constant shaking for 2 hours. The grafted PE samples were taken out from the monomer solution and washed with hot deionized water for 24 hours to remove the homopolymer of AAc.

The amount of grafted PAAc was determined as follows: each PAAc-grafted PE substrate was reacted for 2 hours, at 60° C., with 10 mL of 0.01 M NaOH, and then 5 mL of the supernatant were back titrated with 0.01 M HCl using a Mettler DL21 Titrator (Mettler Instruments, Hightstown, N.J., USA). The grafted amount of PAAc of the AAc-grafted PE substrate was found 36 μg/cm².

The AAc-grafted PE substrates were immersed in 20 mL of aqueous hydrogen peroxide solution (30%) and 4 mL of methanesulfonic acid (99.5%) at 25° C. for 30 min. After the reaction, the samples were immediately washed with cold deionized water, and immersed in an aqueous monomer solution at 25% of NIPAAm. Photografting polymerization of NIPAAm onto the peroxidized sample surfaces was performed by ultraviolet (UV) light irradiation using a 400 W high-pressure mercury lamp for 24 hours. The reaction temperature and irradiation distance between UV light and sample were kept at 20° C. and 18 cm, respectively. The modified surfaces were washed for 3 days with cold deionized water to remove the NIPAAm homopolymers, and dried under nitrogen atmosphere.

To confirm the formation of graft polymerization, the ATR-FTIR was used to evaluate the change of surface functional groups of the PE substrates. From ATR-FTIR spectra, untreated PE samples showed the expected absorptions at 1456 cm⁻¹ for the —CH₂— bending. In the spectra of PNIPAAm-grafted PE surfaces, three absorption bands were observed at 1378 cm⁻¹, 1536 cm⁻¹, and 1648 cm⁻¹. These bands correspond to —C(CH₃)₂ bending, N—H bending (amide II), and C═O stretching (amide I), respectively. Furthermore, the absorbance ratio of the C═O stretching to the —CH₂— bending was used to determine the amount of NIPAAm-grafted chains on the surface layer using a known PNIPAAm amount cast onto PE surfaces as a standard. In these experiments, the optimal grafting amount of PNIPAAm was estimated to be 1.6 μg/cm².

Cultivation and Harvest of HCEC Sheets from Thermo-Responsive Culture Surfaces:

Thermo-responsive PNIPAAm-grafted culture dishes (35 mm in diameter) with an optimal grafting density of 1.6 μg/cm² were used. Prior to the seeding of HCECs, the dishes were subjected to surface sterilization with ultraviolet light for 2 hours in the laminar flow hood.

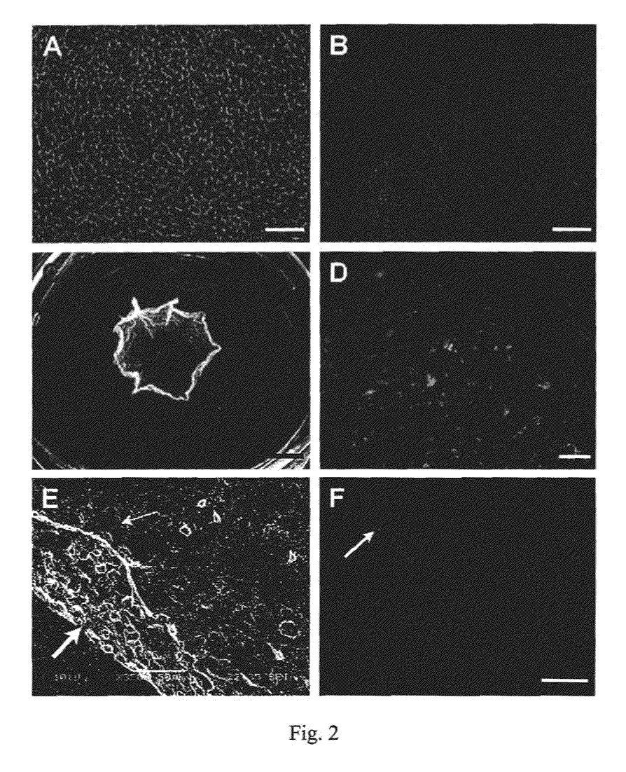

For the purpose of in vivo tracking, HCECs were labeled with PKH26 red fluorescent dye (Sigma-Aldrich) following manufacturer's instructions. Cells were seeded on PNIPAAm-grafted surfaces at a density of 4×10⁴ cells/cm² and incubated under the same conditions as in the above-mentioned HCEC cultivation. Confluence was reached after 1 week of culture. Under a phase-contrast microscope (Nikon, Melville, N.Y., USA), the cultivated HCECs on the hydrophobic PNIPAAm-grafted surfaces in a confluent state possessed a generally polygonal morphology and a high cell density, around 2500 cells/mm², i.e., nearly the same as that found in vivo (FIG. 2A). By a further incubation for 2 weeks in medium, the cultivated HCECs formed a thick layer of extracellular matrix (ECM) beneath the cell sheet. This unique phenomenon of cultivated HCECs possibly indicated the same property of increasing thickness of Descemet's membrane with aging in the human cornea. By lowering the culture temperature to 20° C., the detachment of monolayered HCECs from the switched hydrophilic PNIPAAm-grafted surfaces is a mode of sheet-like movement (FIG. 2B). During the sheet-like movement, each endothelial cell at the leading edge assembles by contracting fan-shaped lamellipodia. In addition, the detached HCEC sheet was harvested as a laminated cell sheet with a gross white paper-like texture (FIG. 2C). The bioengineered HCEC sheet was evaluated by using Live/Dead Viability/Cytotoxicity Kit (Molecular Probes, Eugene, Oreg., USA) following manufacturer's instructions. Results of viability assays showed the monolayered HCECs remained viable after separation from the culture surfaces via a thermal stimulus (FIG. 2D). Under scanning electron microscopy (SEM), polygonal cell morphology was observed throughout the detached HCEC sheet (FIG. 2E). The absence of clear boundaries between these single cells was probably due to the cell contraction caused by detachment at a low culture temperature. Furthermore, the cell sheet had multiple cellular interconnections and abundant deposited ECM. The cell barrier composed of discontinuous tight junction was confirmed by immunohistochemical staining of zonula occludins-1 (ZO-1) on the cell boundary (FIG. 2F). This localization implied that the cultivated HCECs could recruit ZO-1 to the cell borders, i.e., a prerequisite for establishing the passive permeability properties of the endothelial barrier.

### Preparation of Gelatin Hydrogel Discs

Gelatins, prepared through an alkaline processing of bovine bone collagen or an acidic processing of porcine skin collagen, were kindly supplied by Nitta Gelatin (Osaka, Japan). According to information from the supplier, the gelatin samples used as raw materials had IEPs of 5.0 and 9.0, and a weight-average MW range of 3, 8 and 100 kDa, as well as a polydispersity index of 2.0 to 2.5. A gelatin sample with an IEP of “x” and a weight-average MW of “y” kDa was designated as G-x-y. The gelatin hydrogel discs were prepared by solution casting methods as we have described elsewhere [G. H. Hsiue, J. Y Lai, P. K. Lin, J. Biomed. Mater. Res. 61, 19-25 (2002)]. Briefly, after the complete dissolution of gelatin powder in double-distilled water (DDW) at 37° C., an aqueous solution of 10 wt % gelatin (40 mL) was cast into a polystyrene planar mold (5×5 cm², 1.5 cm depth), and air-dried for 3 days at 25° C. to obtain hydrogel sheets. Using a 7-mm diameter corneal trephine device, the hydrogel sheets were cut out to create small gelatin discs (0.4 cm², 700-800 μm thick).

The carrier discs, consisting of gelatins with different isoelectric points (IEP=5.0 and 9.0) and different molecular weights (MW) of 3, 8 and 100 kDa, were subjected to 16.6 kGy gamma irradiation, applied at a dose rate of 0.692 kGy/h; irradiation temperature, 25±1° C., for sterilization. The effect of IEP and MW of raw gelatins (i.e., before irradiation) on the functionality of sterilized discs was studied by determinations of mechanical property, water content, dissolution degree and cytocompatibility.

The mechanical properties of the gelatin carriers were measured with an Instron Mini 44 universal testing machine (Canton, Mass., USA). Dumbbell-shaped specimens were cut from gelatin hydrogel sheets using a punch. The gauge length of the specimens was 10 mm and the width was 5 mm. The thickness of each sample was measured at three different points with a Pocket Leptoskop electronic thickness gauge (Karl Deutsch, Germany) and the average was taken. Experiments were run out at 25° C. and relative humidity of 50% using a crosshead speed of 0.5 mm/min. Results were averaged on twelve independent measurements. Table 1 shows tensile properties of the gelatin hydrogel carriers.

TABLE 1

| | Stress at break | Strain at break | Young's modulus |
|---|---|---|---|
| Sample code | (MPa) | (%) | (MPa) |
| G-5-3 | 4.6 ± 1.4 | 113 ± 28 | 30.7 ± 3.4 |
| G-5-8 | 5.4 ± 1.7 | 109 ± 17 | 35.4 ± 2.9 |
| G-5-100 | 13.1 ± 3.2 | 162 ± 30 | 69.8 ± 6.1 |
| G-9-100 | 11.8 ± 3.5 | 181 ± 32 | 57.5 ± 9.3 |

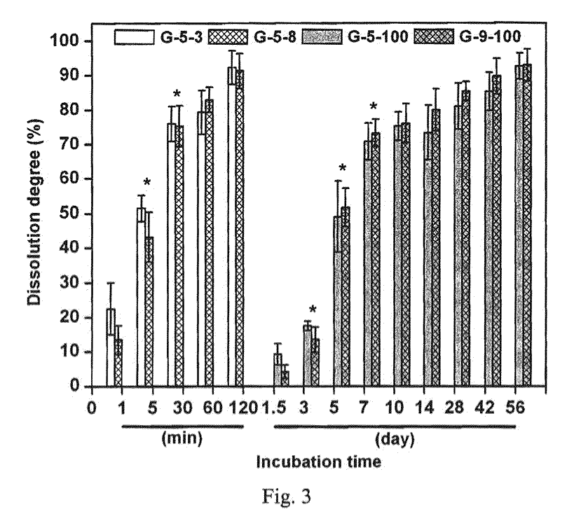

To measure the water content and dissolution degree of the gelatin discs, the samples were first dried to constant weight (Wᵢ) in vacuo and were immersed in BSS at 34° C. (physiological temperature of the cornea) with reciprocal shaking (125 rpm) in a thermostatically-controlled water bath. The swollen hydrogel discs were withdrawn on a filter paper at certain time intervals during the short-term incubation i.e., within 1 day. After removal of excess superficial water, the weight of disc samples at swollen state (Wₛ) was assessed and the water content was defined by ((Wₛ−Wᵢ)/Wₛ)×100. After a long-term incubation (1 day to 2 months), the gelatin discs were dissolved and dried in vacuo again. The dry weight of disc samples after dissolution (W_{d}) was determined and the dissolution degree was calculated as ((Wᵢ−W_{d})/Wᵢ)×100. All experiments were conducted in triplicate. Table 2 shows water content measurements of different types of gelatin hydrogel discs. FIG. 3 shows the time course of dissolution degree of various gelatin hydrogel discs after incubation in BSS at 34° C., wherein an asterisk indicates statistically significant differences (*p<0.05; n=3) for the mean value of dissolution degree compared to value at previous time point.

TABLE 2

| | Immersed time | | | | |
|---|---|---|---|---|---|
| Gelatin disc* | 0 | 5 min. | 60 min. | 360 min | 1440 min |
| G-5-100 | 0% | 37 ± 7.1% | 73 ± 7.1% | 81 ± 5.1% | 90 ± 3.7% |
| G-9-100 | 0% | 40 ± 7.8% | 73 ± 6.4% | 84 ± 3.9% | 89 ± 4.9% |

*G-5-100: IEP = 5.0, MW = 100 kDa; G-9-100: IEP = 9.0, MW = 100 kDa

At each time point, the measured water content of gelatin discs did not show any significant difference between the G-5-100 and G-9-100 groups (p>0.05). This result indicated that the IEP of raw gelatin gives no influence on the water content of gamma-sterilized hydrogel carriers.

As shown in FIG. 3, for each time point, no significant difference was observed in the dissolution degree between G-5-3 and G-5-8 groups, and between G-5-100 and G-9-100 groups (p>0.05). The hydrogel discs prepared with low MW gelatin (3 kDa and 8 kDa) were dissolved for a shorter time period, while the time period of disc dissolution became longer with an increase in the MW of raw gelatin. These findings indicated that the in vitro dissolution rates of gamma-sterilized hydrogel carriers depended heavily on the MW of raw gelatin. In the G-5-3 and G-5-8 groups, the dissolution degree reached a plateau level of approximately 76% within 30 min. These gelatin discs dissolved in physiological solution too fast to be used for cell sheet delivery. In the case of G-5-100 and G-9-100 groups, the dissolution degree had increased by 7 days and continued to increase by about 92% at 56 days. This result suggested that the implanted hydrogel carriers made of high MW gelatin (100 kDa) in the anterior chamber can be dissolved to an extent required for the establishment of close contact between the graft and defective tissues.

Next, the gelatin conditions were optimized by applying the gelatin disc of various molecular weights (MW=3,000, 8,000 and 100,000) and isoelectric points (IEP=5 and 9) into the anterior chamber of the rabbit. Therefore, the triggered tissue responses were monitored by degrees of anterior chamber cell reactions, intraocular pressure and corneal edema. According to our results, gelatins with a negative charge and higher MW possessed the stable mechanical property, appropriate biodegradability, and acceptable biocompatibility.

Irrespective of the IEP of raw gelatin, hydrogel discs prepared with high MW (100 kDa) exhibited a greater tensile strength, lower water content, and slower dissolution rate than those made of low MW gelatin (8 kDa and 3 kDa). From the investigation of cellular responses to the discs, the negatively charged gelatin (IEP=5.0) groups were more cytocompatible when compared with their positively charged counterparts (IEP=9.0) at the same MW (100 kDa). Additionally, in the negatively charged gelatin groups, only a slight increase in pro-inflammatory cytokine expression was observed with increasing MW of gelatin from 3 to 100 kDa. It is concluded that the gamma-sterilized hydrogel discs made from raw gelatins (IEP=5.0, MW=100 kDa) with appropriate dissolution degree and acceptable cytocompatibility are capable of providing stable mechanical support for cell sheet transfer.

### Transplantation of HCEC Sheets Using Gelatin Disc as Carrier

Based on the aforementioned results, the gamma-sterilized hydrogel discs made from raw gelatins (IEP=5.0, MW=100 kDa) having stable mechanical properties, appropriate dissolution degree and acceptable cytocompatibility were therefore selected to carry the thermally detached HCEC sheets. After cell separation from thermo-responsive culture substrates at 20° C., a bioadhesive gelatin disc (7 mm diameter and 700-800 μm thick) was placed on apical surface of the harvested HCEC sheet, and the gelatin-HCEC sheet construct was spontaneously formed by a 5-min incubation at room temperature.

Given that HCECs in vivo possess polarity and pump water from corneal stroma into the anterior chamber, a correct orientation of the transplanted HCECs must be maintained with the apical side facing the aqueous humor in anterior chamber. Accordingly, the detached HCEC sheet was delivered using a 7 mm gelatin disc (700-800 μm thick, MW=100,000, IEP=5) with the HCECs apical side down to correspond to the cell polarity as in vivo (FIG. 1D). Because of high regenerative capacity of rabbit corneal endothelial cells, we also established an animal model capable of mimicking human corneas by treating this type of cells with mitomycin-C (0.1 mg/ml) for 2 weeks to prevent their proliferation and migration [Majmudar P A, Forstot S L, Dennis R F, et al. Topical mitomycin-C for subepithelial fibrosis after refractive corneal surgery. Opthalmology 2000; 107: 89; Vernon R B, Sage E H. A novel, quantitative model for study of endothelial cell migration and sprout formation within three-dimensional collagen matrices. Microvasc Res 1999; 57: 118]. Before transplantation, the central 7 mm of corneal endothelium was removed with a silicone-tipped cannula at the same rabbit in all groups. The gelatin-HCEC sheet construct (the sheet side up) were then inserted carefully into the anterior chambers (HCEC sheet groups) through a 7.5 mm peripheral corneal incision made at 9 o'clock. The corneal wound was closed with two to three interrupted 10-0 nylon sutures and antibiotic ophthalmic ointment was instilled immediately.

After surgery, 1% chlortetracycline hydrochloride ophthalmic ointment (Union Chemical & Pharmaceutical, Taipei, Taiwan, ROC) was immediately applied to the ocular surface. For topical administration of corticosteroids, each rabbit eye received two drops of 0.3% gentamicin sulfate ophthalmic antibiotic solution (Oasis, Taipei, Taiwan, ROC) and one drop of 1% prednisolone acetate ophthalmic steroid suspension (Pred Forte, Allergan, Westport, Co. Mayo, Ireland) four times a day during the follow-up period of 3 months. The control groups included a traumatized cornea without a transplant (wound groups) and with a gelatin disc only (gelatin groups) were also treated with ophthalmic ointment and topical steroids the same as the HCEC sheet groups. In HCEC sheet groups, after surgery, slit-lamp biomicroscopy revealed that the anterior chamber was filled up with the gelatin-HCEC sheet construct. Moreover, an intact, round-shaped layer of HCECs was positioned onto the denuded corneal posterior surface. The following day, severe corneal swelling was noted, and persisted until completion of the experiment in wound and gelatin groups. At postoperative 2 weeks, the gelatin discs largely dissolved and HCEC sheet was attached onto the denuded surface of Descemet's membrane in the HCEC sheet groups. The swollen cornea returned to clarity and a nearly normal corneal thickness after implantation of a HCEC sheet 4 weeks postoperatively.

Histological examination under light and fluorescent microscopy revealed that, after surgery for 2 weeks, the implanted HCECs labeled with PKH26 red fluorescent dye remained attached, subsequently forming tight junctions on a flat mount and cross section. The corneal thickness of traumatized corneas with transplanted HCEC sheet improved more significantly than that of the control groups during the first postoperative 2 weeks. All corneas in the control groups did not return to normal during the follow-up period of 3 months.

In summary, the present invention described a novel cell therapeutic method for HCEC loss, by mass cultivating HCECs from adult human corneal donors, harvesting HCECs as a cell sheet after detaching from a thermo-responsive PNIPAAm-grafted surface and delivering HCECs with a negatively charged, high molecular weighted gelatin disc. The transplanted HCEC sheet was integrated into the denuded corneas, with the returned corneal clarity after transplantation indicating the function of the transplant. Results of the present invention demonstrated the feasibility of transplanting HCEC sheet for corneal endothelial cell loss and as a possible alternative to PK.

It is conceivable that the novel cell therapeutic method of the present invention also provide a new approach for reconstructing corneal epithelium in a patient.

1. A method for reconstructing corneal endothelium in a patient comprising implanting a biopolymer-bioengineered cell sheet construct into an anterior chamber of a cornea of the patient, wherein the construct comprises a biopolymer carrier which is bioresorable and deformable, wherein said biopolymer carrier is made of gelatin having a weight-average molecular weight of 50,000 to 100,000 Dalton, and an isoelectric point of 5-9; and a bioengineered cell sheet comprising a monolayer or multilayer of interconnected human corneal endothelial cells with substantially uniform orientation, wherein said bioengineered cell sheet is attached to a surface of said carrier with apical surfaces of the cells facing said carrier, wherein the biopolymer-bioengineered cell sheet construct is implanted into the anterior chamber with basal surfaces of said cells of said bioengineered cell sheet contacting a posterior surface of the cornea.

2. The method of claim 1 further comprises removing unhealthy endothelium from the posterior surface of the cornea of the patient before said implanting

3. The method of claim 1, wherein said implanting comprises forming an incision at a limbus of the cornea; inserting the biopolymer-bioengineered cell sheet construct through the incision into the anterior chamber; and closing the incision by suturing, so that the biopolymer-bioengineered cell sheet construct is enclosed in the anterior chamber, wherein the carrier will become swollen by aqueous humor in the anterior chamber, creating a pressure pressing the bioengineered cell sheet against the posterior surface of the cornea, and the carrier is eventually biodegraded in situ while an endothelial sheet is regenerated on the posterior surface of the cornea.

4. The method of claim 2, further comprises removing unhealthy endothelium from the posterior surface of the cornea before inserting the biopolymer-bioengineered cell sheet construct into the anterior chamber.

5. The method of claim 1, wherein said bioengineered cell sheet further comprises an extracellular matrix distributed at basal surfaces of said cells.

6. The method of claim 1, wherein said biopolymer carrier is made of poly(amino acids), gelatin, collagen, polysaccharide, hyaluronan, chitosan, alginate, agarose, poly(α-hydroxy acid), or a mixture thereof.

7. The method of claim 1, wherein said biopolymer carrier is made of gelatin.

8. The method of claim 1, wherein said gelatin has a weight-average molecular weight of about 100,000 Dalton, and an isoelectric point of about 5.

9. The method of claim 1, wherein said gelatin is negatively charged.

10. The method of claim 1, wherein said biopolymer carrier has a thickness of 0.5-1.0 mm and a diameter of 5-10 mm, and has a water content of 10-90%, based on the dry weight of the biopolymer carrier.

11. The method of claim 10, wherein said biopolymer carrier has a water content of less than 40%, based on the dry weight of the biopolymer carrier, when said bioengineered cell sheet is attached to the surface of said carrier, and said carrier becomes swollen and the water content thereof becomes at least 1.5-fold when the carrier is surround by an aqueous solution for a period of 5 minutes or more.
